(19) United States
(12) Patent Application Publication
(10) Pub. No.: US 2022/0040215 A1
(43) Pub. Date: Feb. 10, 2022

(54) MUSCLE TREATMENT COMPOSITION AND METHOD OF MAKING SAME

(71) Applicant: Organic Medical Ventures, L.L.C., Norman, OK (US)

(72) Inventors: Gary L. Rayburn, Norman, OK (US); Robert D. Maurer, Miramar Beach, FL (US); Marvin A. Cuchens, Santa Rosa Beach, FL (US)

(73) Assignee: Organic Medical Ventures, L.L.C.

(21) Appl. No.: 17/508,066

(22) Filed: Oct. 22, 2021

Related U.S. Application Data
(62) Division of application No. 14/677,215, filed on Apr. 2, 2015, now abandoned.
(60) Provisional application No. 61/975,558, filed on Apr. 4, 2014.

(51) Int. Cl. A61K 31/7076; A61K 47/12; A61K 36/53; A61K 31/60; A61K 33/00; A61K 38/39; A61K 36/28; A61K 9/00; A61K 31/122; A61K 31/20; A61K 31/385

(52) U.S. Cl. CPC A61K 31/7076; A61K 47/12; A61K 36/53; A61K 31/60; A61K 33/00; A61K 31/385; A61K 36/28; A61K 9/0014; A61K 31/122; A61K 31/20; A61K 38/39

(57) ABSTRACT

A topical treatment composition for treating muscle cramps, muscle stiffness, muscle pain or muscle spasms. The treatment composition includes a middle (mid) chain fatty acid (MCFA). A method of making the topical treatment composition is also disclosed herein.

### CROSS-REFERENCE TO RELATED APPLICATIONS

The present application is a divisional of U.S. patent application having U.S. Ser. No. 14/677,215, filed Apr. 2, 2015, which is a conversion of U.S. Provisional Application having U.S. Ser. No. 61/975,558, filed Apr. 4, 2014, which claims the benefit under 35 U.S.C. 119(e), the disclosure of which is hereby expressly incorporated herein by reference.

### STATEMENT REGARDING FEDERALLY SPONSORED RESEARCH OR DEVELOPMENT

Not applicable.

### BACKGROUND

The anatomy, biochemistry, and physiology of muscle contractions are fairly well established in the medical field. (J E Hall, Guyton & Hall Textbook of Medical Physiology, Sanders Elsevier, Philadelphia, PA (2011); E Newsholme, T Leech, Functions & Biochemistry in Health & Disease, Wiley-Blackwell, West Sussex, UK (2009)) However, topical application(s) of the current understanding in these disciplines have not been fully appreciated or applied. The purpose of the currently disclosed and claimed inventive concept(s) relates to methods and compositions for topically treating muscles to promote energy usage under a variety of stimuli for the purpose of improving and maintaining health and fitness.

The currently disclosed and claimed inventions have potential applications for numerous muscle conditions, including those referred to as fatigue, cramps, tiredness, exhaustion, lethargy, listlessness and weakness. Additional applications which are related to the noted conditions pertain to energy requirement(s) under normal and abnormal activities during use/exercise. These conditions and their associated symptoms may occur in normal individuals, as well as in associations with numerous muscular symptoms and diseases (myopathies).

One potential application of the currently disclosed and claimed inventions is with muscular fatigue. This is often associated with a lack of cellular energy and more specifically, depletion of adenosine triphosphate (ATP). Physiologically, such a lack of ATP at the biochemical level is also associated with a feedback to/from neurons leading to peripheral fatigue. Hence one goal is to provide topical nutritional treatments for the relief of muscular fatigue.

One potential application of the currently disclosed and claimed inventions is with cramps that are associated with various activities or conditions and may also be known by an assortment of alternative names; e.g., a Charlie horse, a muscle spasm (aka a pulikat), night time (or nocturnal) cramp, menstrual cramp, heat cramp, writer's cramp (physical, not mental), runner's cramp, and swimmer's cramp. The most commonly involved muscle groups are the lower leg/calf (gastrocnemius & Soleus), the thigh (quadriceps & hamstrings), feet (intrinsic muscles of the feet & short flexors, to include individual toes), hands (intrinsic muscles, including individual fingers), arms, abdomen, and along the rib cage (intercostal muscles), although other muscle groups may be affected, albeit to a lesser extent. Regardless of the muscle group, a true skeletal muscle cramp typically exhibits a forceful, involuntary, painful contraction of the muscle. A cramp tends to be debilitating/incapacitating and may last for a few seconds, minutes or longer periods. They may frequently or intermittently reoccur with or without any established pattern in frequency. They may be associated with various medical treatments/drugs or, as noted above, various conditions/diseases.

In addition, muscle contractures may result when the muscles are unable to relax for an even more extended period than a common muscle cramp. This form of a cramp results from constant spasms wherein it is thought that the nerves are inactive. Contractures can also result from inherited or acquired conditions, which affirm the role of cellular energy in fatigue and cramps. On one hand, inherited McArdle's muscular dystrophy (characterized by an inherited defect of the breakdown of glycogen to sugar), is generally characterized by variable degrees of muscle weakness and degeneration from an inability to produce energy. On the other hand an acquired condition such as hyperthyroid myopathy (a muscle disease associated with an overactive thyroid) represents yet another variant wherein too much energy is utilized due to an overactive thyroid. Again, these and all general forms of muscular fatigue and/or cramps are associated with a depletion of ATP, “the” basis for chemical cellular energy and life in general, leading to insufficient levels of ATP for continued muscle fiber contraction or relaxation. The latter results in a possible muscular rigor (cramp) from too much adenosine diphosphate (ADP: see below; the phosphate depleted, expended form of ATP).

Another form of (dystonic) cramps may occur when muscles that are not needed for an intended movement are stimulated to contract. Muscles that are affected by this type of cramping include those that ordinarily work in the opposite direction of the intended movement, and/or others that exaggerate the movement. These types of cramps usually affect small groups of muscles (e.g., eyelids, jaws, neck, larynx, etc.). The hands and arms may be affected during the performance of repetitive activities such as those associated with handwriting (writer's cramp), typing, playing certain musical instruments, and many others. Each of these repetitive activities may also eventually produce true cramps from muscle fatigue.

However, the vast majority of true cramps occur in two major demographics.

The night-time (nocturnal) cramping demographic is particularly common among the elderly. About a third of people over 60 years of age and half of individuals over 80 suffer from this condition. This represents greater than 20 million people in the U.S. Of these, forty percent experience more than three attacks per week. (T Wallimann, M Tokarska-Schlattner, U Schlattner, Unknotting night-time muscle cramp: a survey of patient experience, help-seeking behavior and perceived treatment effectiveness, Amino Acids (2011) 40:1271-1296; F Blyton, V Chuter, J Burns, Unknotting night-time muscle cramp: a survey of patient experience, help-seeking behavior and perceived treatment effectiveness. J. Foot Ankle Res (2012); 5:1-8; F Hawke, V Chuter, J Burns, Factors associated with night-time calf muscle cramps: A case-control study. Muscle Nerve (2012)).

An even larger demographic is represented by more than millions of individuals involved in athletics or jobs requiring rigorous physical activities. For example, there are approximately 7.2 million college and high school athletes in the U.S. This number does not include the millions of amateur and recreational athletes or the numerous professionals. In the US; this includes, as examples, 24 million golfers, 24 million tennis players, 42 million health club memberships and 0.65 million marathon runners. Collectively approximately 43% of the more than 300 million Americans exercise on a regular basis. Research has shown that a high percentage of all athletes experience cramping at some time. Muscle cramping tends to be very painful, typically requires the athlete to cease further activity, and can occur acutely and/or chronically in athletes in a wide variety of performance levels and activities. These occurrences can drastically affect the outcome of a competitive sport and clearly impacts an athlete's personal goal(s) in training and performance. Similarly, performance can be significantly affected by cramps in individuals with physically demanding jobs in the work force. No one is immune from cramps; i.e., virtually everyone will sooner or later experience cramps.

The most common approach to relieve a cramp upon onset is to use massage. A cramp occurs when muscles are unable to relax properly and usually involves one of the two opposing muscles that become locked in a rigor configuration. However, an attempt to force a cramped muscle by contracting the opposing muscle can tear muscle tissue and worsen the pain, i.e., even with massage, care must be used and the muscle must be allowed to recover. Thus there exists a long-felt need for topical compositions that effectively relieve a muscle cramp or the symptoms associated with a cramp and/or prevent the reoccurrence of a cramp. This is not to exclude other types of cramps, such as menstrual cramp.

The scope of the presently claimed and disclosed inventive process is to provide chemical energy to muscles. One of the applications of this technology is to relieve the rigor of a cramp (see below). Scientific studies have clearly demonstrated that hypo-hydration (dehydration) or electrolyte imbalance is not the cause of cramps. Furthermore, IV fluids, bananas, mustard, pickle juice, etc., do not relieve cramps. (K C Miller, G Mack, K L Knight, Electrolyte and Plasma Changes After Ingestion of Pickle Juice, Water, and a Common Carbohydrate-Electrolyte Solution, J Athletic Training (2009) 44:454-461; K C Miller, G W Mack. K L Knight, J T Hopkins, D O Draper, P J Fields, I Hunter. Three Percent Hypohydration Does Not Affect Threshold Frequency of Electrically Induced Cramps. Med Sci Sports Exerc (2010) 42:2056-2063; K C Miller, G W Mack, K L Knight. Gastric Emptying After Pickle-Juice Ingestion in Rested, Euhydrated Humans. J Athletic Training (2010) 45:601-608; K C Miller, M S Stone, K C Huxel, J E Edwards. Exercise-Associated Muscle Cramps: Causes, Treatment, and Prevention. Sports Health (2010) 2:279-283). The presently disclosed and claimed inventive concepts specifically utilize a method using a scientific/medical approach to topically treat and/or inhibit muscle cramps and the associated pain, spasms and stiffness.

Another application of a topical muscle energy supplementation is in physical therapy and in the rehabilitation or recovery of muscles post trauma, surgery, degeneration after long term debilitation, excessive exercise (e.g., Underperformance Syndrome, or UPS, associated with overtraining), muscle injury, etc. For example, muscles that are immobilized, as by a plaster cast following fracture of a long bone, tend to waste rapidly through shrinkage of the muscle fibers. Topical applications to the affected limb(s) with a formula to provide chemical energy to facilitate muscle recovery, regeneration or therapy during muscle-strengthening exercises clearly have potentially practical and medical benefits. Applications in physical therapy may also include the treatment of certain muscle myopathies, such as muscular dystrophy (e.g., in two common forms, Duchenne and Becker).

Another application is with phantom pain that is often associated with loss of limb. Either relief from nerve stimulation associated with the phantom pain (e.g., additional ATP to relieve muscular activity associated with overstimulation of the nerves) and/or the potential benefit for additional energy from topical application to assist in motor functions of the affected limb(s) using the currently disclosed and claimed inventions has potential benefits for amputees.

Yet another application is with massage therapy to relieve, for example, acute muscle soreness, stiffness, tightness, fatigue or contraction.

Such a topical application as currently disclosed in the claimed inventions also has ramifications for athletic performance and the physiology associated therein, with applications before, during or after any one of numerous athletic events. Clearly, different training techniques may impact potential uses, compositions, delivery, etc., of these methods. (G Whyte (ed), The physiology of training, (2006) Elsevier, UK). Along these lines, although there are multiple fuel sources available, there are multiple factors involved that determine which energy source is utilized in muscle; inter alia, the type of muscle, amount of stored fuel, conditioning, intensity and duration of exercise, blood flow and O₂ availability, etc. Furthermore, there clearly are other potential applications, such as altitude training, as well as with delayed onset muscle soreness (DOMS; aka muscle fever). Nevertheless, the methods as currently disclosed in the claimed inventions provide approaches to address a multitude of issues in exercise physiology.

In addition, the topical treatment described herein is for some chronic diseases and/or disabilities; e.g., muscular dystrophy, fibromyalgia (e.g., in exercise regimens for treatment), combat-fatigue, assist in conditioning for rheumatic conditions, etc. Some muscular dystrophies, including Duchenne's and Becker's, are a large group of diseases, many of which are hereditary or result from genetic mutations, where the muscle integrity is disrupted. Many lead to progressive loss of strength and decreased life span. In general a large proportion of neurological disorders also lead to problems with movement. Some examples of central (or upper motor neuron) disorders include cerebrovascular accident (e.g., a stroke), Parkinson's disease, multiple sclerosis (MS), Huntington's disease (Huntington's chorea) and Creutzfeldt-Jakob disease. Myasthenia gravis and Lambert-Eaton syndrome, examples of neuromuscular junction disorders, and muscular dystrophies or inflammatory myopathies (e.g., polymyositis), examples of primary muscular (myopathic) disorders, may also benefit from topical applications to generate energy for muscles. Applications in the treatment of the symptoms associated with other conditions may also exist; e.g., Chronic fatigue syndrome (associated with prolonged tiredness), Fibromyalgia (associated with widespread musculoskeletal pain and fatigue), Myositis (associated with degeneration of muscle tissue), and certain viral infection.

In addition, the topical treatment described herein is for muscular strain, generally defined as an injury to a part of the body caused by overexertion or twisting muscle awkwardly. There clearly is efficacy in providing ATP in any cellular process, including regenerative processes in muscle repair.

In general, the intent of the topical applications described herein is to provide topical application of a source for either direct or indirect energy (i.e., direct application of ATP or a means to rapidly regenerate/generate ATP) for muscles, to directly provide such an energy source without the dilutional effects (after oral intake, gastrointestinal adsorption, and venous distribution), and to minimize the impact of the liver's effects in metabolism/catabolism/detoxification. The premise is that direct adsorption to the affected site will provide more immediate and effective relief through topical delivery, as opposed to oral delivery that is diluted, has potential affects from the first passage through the liver, will be delayed in distributing/delivering a sufficient amount/concentration to the affected site (i.e., temporal issues) and may generate potential side effects to other organs/parts of the body.

### DETAILED DESCRIPTION OF THE DISCLOSURE

The currently disclosed and claimed inventive concept(s) relates to compositions and methods of making and using said compositions for topically treating muscles to promote energy usage under a variety of stimuli (or lack thereof) for the purpose of improving and maintaining normal muscle health and fitness, for the treatment of cramps, or for the treatment of certain muscle conditions (e.g., strains, fatigue, etc.), diseases and/or myopathies. The intent is to supplement and/or metabolically assist key components in the cellular process(es) to elevate ATP itself or the substrates involved in regenerating or generating additional ATP. These processes will be applicable not only in the basic mechanics of exercise, but also in regeneration processes after exercise, to relieve cramps (wherein ATP is depleted and the muscle remains in a contracted, rigor, state), and in therapies for normal muscle regeneration or in degenerative processes (e.g., long term bed rest or certain muscular myopathies). This utilizes the fact that ATP fuels virtually all energy requiring processes, including muscle contraction and regeneration. Obviously, energy production after topical application has implications for supplemental energy for cells other than muscles and cells (e.g., regeneration of cells after burns, surgical wounds, etc.).

With respect to muscle activity, the mechanics of contraction are generally described as: 1) nerve stimulation leading to calcium release and the relaxation of the attachment of myosin to actin within a muscle fiber, 2) the subsequent binding of ATP to myosin, 3) a cascade of biochemical events that release free phosphate and enables the remaining ADP (bound to myosin) to stimulate the myosin to physically move the actin, and 4) actin proteins at opposite ends (within a sarcomere, within the muscle fiber) are brought together towards the center, resulting in contraction. It is generally recognized that 1) without additional ATP, the muscle remains contracted and 2) a depleted supply of ATP is the cellular basis for a cramp/rigor in a muscle. (J A Rathmacher, J C Fuller, S M Baier, N M Abumrad, H F Angus, R L Sharp, Adenosine-5′-triphosphate (ATP) supplementation improves low peak muscle torque and torque fatigue during repeated high intensity exercise sets, J International Society Sports Nutrition (2012) 9:48-55; F. Martini, Fundamentals of Anatomy and Physiology, Ch. 10, Benjamin-Cummings Publishing Co, (2012) NJ; D. DeWitt, Physics of Sports: http://www.phys.washington.edu/%7Ewilkes/post/ternp/phys 208, Skeletal Muscle, v2.1, (2005); HOW MUSCLES WORK, http://health.howstuffworks.com/wellness/diet-fitness/exercise/sports-physiology6.htm; M W Berchtold, H Brinkmeier, M Muntenerner, Calcium Ion in Skeletal Muscle: Its Crucial Role for Muscle Function, Plasticity, and Disease, Physiological Reviews, (2000) 80:1216-1265). The premise of the presently disclosed and claimed inventive process is to topically supply additional ATP.

On one hand, additional, topically supplied, ATP will provide a means to sustain normal muscle activity (e.g., improve endurance, improve recover after exercise and prophylactically reduce the incidence of cramps). On the other hand, additional ATP will provide relief from muscular fatigue or a cramp in a contracted muscle depleted of ATP or additional energy to dysfunctional muscles (e.g., atrophied muscles, strained muscles, myopathies, etc.).

To maintain/sustain ATP for continuous movement under normal, non-cramped conditions, cells initially use the limited intracellular store of ATP and then the intracellular store of creatine to provide a phosphate to ADP (generated during the “power stoke” in muscle contraction). This regenerates ATP from ADP. However, intracellular stores of ATP and creatine are limited and the cell must resort to generating new ATP. Initially, blood glucose and then liver/muscle glycogen are used, but these resources are also quickly used within minutes. Subsequently cells utilize fatty acids and eventually proteins, which supply even greater amounts of ATP for energy. Note that collagen is a protein store initially used as a protein source of energy; other proteins are typically used as a last resort (e.g., during starvation). Throughout these events, numerous enzymes, cofactors, substrates, intermediates, etc., are utilized in the biochemical processes to utilize existing ATP stores, regenerate ATP from ADP and generate additional ATP. There are also additional unique energy resources (e.g., lactate, glutamine, and ketone bodies; see below), as well as additional factors that mitigate the after-effects of muscle use/exercise.

The presently disclosed and claimed inventive concept(s) is directed at applying these basic concepts to muscles by topically supplying: 1) supplemented ATP, 2) supplemented creatine (e.g., through de novo synthesis) to regenerate ATP, 3) supplemented glucose and/or glycogen as energy sources to generate additional ATP, 4) collagen (or hydrolyzed collagen, mixture of various peptides, etc.) as an additional substrate to generate ATP, 5) unique fatty acids as a direct energy source to generate additional ATP or to alleviate the stress issue of muscle usage (e.g., the lactic acid burn from exercise) and 6) a supplement of one or more compounds (inter alia coenzyme Q10, lipoic acid/liponate, NADPH, FADH₂ pyruvate, citrate, fumarate, malate or succinate) as factors/cofactors/substrates/intermediates involved in the biochemical pathway(s), to generate/amplify production of ATP or mitigate issues involved with muscle contractions. Each of these compounds are either naturally found in cells or naturally utilized by cells. The premise is to use these and other compounds that are important for direct chemical energy, regenerating/generating chemical energy for muscle contraction and/or alleviating adverse muscle conditions.

Adenosine Triphosphate (ATP) is, above all other molecules, the primary carrier of chemical energy in cells, serving to transfer high energy phosphate groups from energy-yielding to energy-requiring processes. Likewise, ATP provides energy to muscle cells by way of its phosphate (PO₄). As ATP supplies energy, it loses a phosphate group; ATP→ADP (tri- to di-phosphate forms). The key to all sustained muscle activity is the immediate supply of available ATP. Along these lines the currently disclosed and claimed inventive concept(s) relate to the topical application of basic biochemical processes involved in energy utilization and ATP production for muscles as outlined below.

After exercise begins, the muscle begins to immediately use its available stores of ATP. The existing ATP is quickly converted to ADP; this is sufficient for only approximately 0.5 second of muscle contraction. In one aspect, the currently disclosed and claimed inventive concept relates to topically supplementing ATP to the muscle group(s) under consideration for treatment. Note that the efficacy of orally supplementing ATP and its beneficial impact on athletic performance has been demonstrated (Rathmacher, et al.). The efficacy of using free ATP is clear, yet the impact of dilutional effects, as referenced, clearly impacted oral dosing.

ATP compounds for topical use in the compositions and methods described herein include all and any ATP compounds, analogs, or derivatives and pharmaceutically-acceptable hydrates and salts thereof that are suitable for being metabolized in a similar manner to endogenous ATP. Preferably, compositions of the present invention contain a concentration of ATP wherein the amount of ATP delivered to the affected cells after topical application is greater than that found endogenously. A composition of the present invention may contain between 0.01% and 90% (w/v) of ATP. Preferably, the ATP is between 1% and 10% (w/v) of the composition, inclusive of the endpoints. In other embodiments, the ATP has a concentration of at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In a particular embodiment, ATP has a concentration of between 0.6% and 2% (w/v); for example, a concentration of about 0.66% or 1.3% (w/v) of the composition.

After immediately available ATP is used, the available cellular stores of creatine are then utilized to regenerate ATP by providing a phosphate group to the ADP (the phosgene system). The de novo synthesis of creatine (as disclosed below) to treat cramps is applied below, as well as for the use of creatine in non-exercise, non-cramp related issues as described above, e.g., with myopathies.

In particular, the presently disclosed and claimed inventive concept(s) also relates to method(s) to treat muscle symptoms using creatine which is formed by de novo synthesis from its precursors provided topically. Creatine is a phosphate containing compound which is normally stored in the muscles. It is primarily synthesized by the liver and kidney and then transported by the blood to skeletal muscles. Approximately 95% of the body's creatine (half obtained from endogenous synthesis and the other half from dietary sources) is stored in skeletal muscles where concentrations may reach 20-35 mM (or approximately 0.26%-0.46%). It cannot be used directly to power muscle contraction. However it does serve a key role in muscle contraction by the transfer of its phosphate group to ADP (which is in higher amounts in a contracting muscle) to regenerate ATP as follows: Creatine phosphate+ADP+H⁺→Creatine+ATP. The newly regenerated ATP again then acts as a direct chemical energy source for contraction. (The creatine kinase system and pleiotropic effects of creatine, T Wallimann, M Tokarska-Schlattner, U Schlattner, Amino Acids (2011) 40:1271-1296; R Cooper, F Naclerio, J Allgrove, A Jimenez, Creatine supplementation with specific view to exercise/sports performance: an update, J Int Soc Sports Nutr, (2012) 9:33-44; M Wyss, R Kaddurah-Daouk, Creatine and Creatinine Metabolism, Physio Rev (2000) 80: 1108-1213; AM Persky, GA Brazeau, Clinical Pharmacology of the Dietary Supplement Creatine Monohydrate, Pharmacol Rev (2001) 53:162-176). In a cramp, it is predicted that both ATP and the creatine will be utilized virtually in toto. The premise in the presently disclosed and claimed inventive concept is to supplement existing cellular creatine by exogenous means (see below for the de novo synthesis of creatine) to synthesize creatine via topical application to muscles in order to regenerate ATP from ADP. It is predicted that supplemental ATP and creatine will have an additive and/or synergistic effect in providing ATP.

Although the phosgene system is immediately available and efficient, it is not sustainable. Only 1 ADP/creatine molecule can be regenerated to one ATP and is sufficient for approximately an additional 8-10 seconds of continued muscle contractions. In order to sustain further muscle contractions, an infusion of additional ATP is required.

In particular, the presently disclosed and claimed inventive concept(s) also relates to method(s) to topically treat muscles using glucose. The initial infusion of additional ATP normally comes from glycolysis. This initially utilizes the immediately available glucose from blood and interstitial spaces. Glycolysis converts glucose (C₆H₁₂O₆) into pyruvate (CH₃COCOO⁻+H⁺) in a definite sequence of ten reactions involving ten intermediate compounds. This anaerobic metabolism of one molecule of glucose to two molecules of pyruvate has a net yield of two molecules of high energy ATP; pyruvate in turn enters the citric acid cycle (aka the tricarboxylic acid cycle (TCA cycle) or the Krebs cycle) to generate additional ATP (approximately 30) and is sufficient to sustain an additional 1.3-1.6 minute of muscle contraction. Preferably, compositions of the present invention contain a concentration of glucose wherein the amount of glucose in the composition may contain between 0.01% and 90% (w/v) of glucose. Preferably, the glucose is between 1% and 10% (w/v) of the composition, inclusive of the endpoints. In other embodiments, the glucose is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In a particular embodiment, the glucose has a concentration of about 3.5% (w/v).

In particular, the presently disclosed and claimed inventive concept(s) also relates to method(s) to topically treat muscles using glycogen. To further sustain muscle contraction, the muscle must subsequently resort to stored fuels. Within muscles, the immediately available resource is in the form of glycogen. Glycogen is a multi-branched polysaccharide (long chained carbohydrates) that is made and stored primarily in the cells of the muscle and the liver; approximately 50% in each. Glycogen comprises approximately 1% of muscle mass and forms an energy reserve that can be quickly mobilized to meet a sudden need for glucose. Glycogen is cleaved from the non-reducing ends of the chain by the enzyme, glycogen phosphorylase, to produce monomers of glucose-1-phosphate, which are then converted to glucose 6-phosphate (i.e., glucose) by phosphoglucomutase. Unlike liver cells, muscle cells cannot export glucose converted from glycogen, i.e., it is preferentially used for energy. Again, the end result is pyruvate, which can be directed into lactate or into the citric acid cycle via formation of Acetyl-CoA, depending on the availability of oxygen. Again, there is amplification in ATP produced from each glucose molecule generated from glycogen. In one particular embodiment, the composition comprises a quantity of glycogen (or a suitable substitute thereof) between about 0.1% and about 90% (w/v). Preferably, the glycogen is between 0.5% and 10% (w/v) of the composition, inclusive of the endpoints. In other embodiments, the glycogen is at least 0.1%, 0.25%, 0.5%, 0.75%, 1%, 2%, 3%, 4%, 5%, 6%, 7%, 8%, 9%, 10%, 12.5%, 17.5%, 20%, 25%, 30%, 40%, 50%, 60%, 70%, 80%, or 90% (w/v) of the composition. In one particular embodiment, the glycogen has a concentration of between 1% and 5% (w/v).

In particular, the presently disclosed and claimed inventive concept(s) also relates to method(s) to treat muscles using collagen. In normal exercise regiments, protein stores offer another tier for energy utilization. One immediately available source is collagen (a multi-chained protein). Muscles contain approximately 6% by weight of collagen and collagen represents 25-35% of all proteins in the body. The catabolism (breakdown) of proteins, and in this case collagen, in muscles is complex and not as energy efficient (as is the case with the MCFA; see below). Nonetheless protein catabolism leads to additional entry into the citric acid cycle via Acetyl-CoA complexes that are formed and consequentially additional ATP.

Collagen for use in the compositions and methods described herein include all and any collagens, analogs (e.g., gelatin), or derivatives (e.g., hydrolyzed collagen, palmitoyl pentapeptide (Matrixyl), or other synthetic amino acids or peptides). The rational for using smaller peptides for a topical application is the difficulty in adsorbing the larger molecules of collagen or other proteins transdermally. As such hydrolyzed collagen and pharmaceutically-acceptable hydrates and salts thereof or a suitable substitute represent a mixture of peptides normally present in catabolism of collagenase that are suitable for being metabolized in a similar manner to endogenous collagen for the de novo synthesis of ATP. Hydrolyzed collagen is also called collagen hydrolysate, collagen peptide, gelatine, gelatine hydrolysate and hydrolyzed gelatine. The hydrolysis process results in reducing the collagen proteins of about 300,000 Da into small peptides having an average molecular weight between 2000 and 5000 Da. It is widely and safely used in cosmetics. Hydrolyzed collagen contains 8 out of 9 essential amino-acids, including glycine and arginine, two amino-acid precursors necessary for the biosynthesis (see below) of creatine. As such the use of supplemented glycine and arginine from creatine may also impact the regenerative properties of cells by providing metabolites for the synthesis of creatine (see below), which is involved in converting ADP to ATP.

A composition of the present invention may contain between 0.1% and 90% (w/v) of collagen or a suitable derivative or substitute thereof. Preferably, the collagen is between 1% and 10% (w/v) of the composition, inclusive of the endpoints. In other embodiments, the collagen is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one aspect, the composition contains a hydrolyzed collagen that is has a concentration of between 1% to 15% (w/v) of the composition; e.g., a concentration of about 5% or 10% (w/v).

In particular, the presently disclosed and claimed inventive concept(s) also relates to method(s) to treat muscles using Medium (Mid) Chain Fatty Acids (MCFAs). MCFAs, (some of which are contained in the medium chain triglycerides or MCTs), are a class of fatty acids or lipids that are saturated fatty acids (i.e., they are straight chained molecules of carbons with no double bonds, branching, or side groups) with a carbon chain length of C6, C8, C10 or C12, herein referred to as C6 fatty acid (aka caproic/hexanoic acid and salts thereof)), C8 fatty acid (aka caprylic/octanoic acids and salts thereof), C10 fatty acid (aka capric/decanoic acids and salts thereof), and C12 fatty acid (aka lauric/dodeconic acids and salts thereof), respectively.

In contrast to other fatty acids which require facilitated adsorption and transport across the gut, as well as across cellular membranes, MCFAs are easily and directly absorbed and, equally important, quickly and preferentially utilized for energy. All cells can use MCFAs for energy and are so efficient in doing so that MCFAs are not shuttled into fat. In the currently disclosed and claimed inventive processes, MCFAs will be used for several purposes. Specifically: 1) MCFAs will potentially participate in the delivery system, e.g., CoQ10 will dissolve in the MCFA (see below); note that other components in the formulation (see below) may also contribute to the delivery of the various components. 2) MCFAs are potential penetration enhancers (see below) and as such may facilitate the rapid adsorption of components through the skin. And 3) MCFAs will serve as a ready supply of additional ATP.

Specifically, when biochemically compared, the MCFA molecules provide even greater yields of ATP than other metabolites; on a per unit basis, fats provide more than twice the energy than either carbohydrates or proteins. For example, according to the formula (n−1)*14+10−2=total ATP; where n=½ the chain length of an even numbered, saturated hydrocarbon, the potential number of ATPs generated from each of the MCFAs is C6=36; C8=50; C10=64; C12=78. The benefit of MCFAs in energy (ATP) production is its direct uptake and preferential utilization within the cell, as well as the additional ATPs that are generated. (Physiological Effects of Medium-Chain Triglycerides: Potential Agents in the Prevention of Obesity, M-P St-Onge, P J H Jones, Am Soc Nutritional Science (2002) 32:329-332). MCFAs are topically well tolerated and are widely and safely used in the cosmetic industry.

MFCAs for use in the compositions and methods described herein include all and any MFCAs, analogs, or derivatives and pharmaceutically-acceptable hydrates and salts thereof that are suitable for being metabolized in a similar manner to endogenous MFCA for the de novo synthesis of ATP. Examples of MFCAs include, C6, C8, C10, and C12 saturated fatty acids. Preferably, compositions of the present invention contain a concentration of MFCA (or a combination of MFCAs) wherein the amount of MFCA (or combination thereof) delivered to the affected muscle cells after topical application is greater than that found endogenously (which is typically very low due to its rapid utilization for energy). A composition of the present invention may contain between 0.01% and 90% by weight (w/v) of MFCA. Preferably, the MFCA is between 1% and 10% (w/v) of the composition, inclusive of the endpoints. In other embodiments, the MFCA is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90%, by weight (w/v) of the composition. In one aspect, the MCFA is a C8 fatty acid or a C12 fatty acid, and the C8 or C12 fatty acid does not exceed 10% (w/v) of the composition. In another aspect, the MFCA is a combination of C8 and C12 fatty acids, wherein the combination does not exceed 20% (w/v) of the composition.

The intent of the presently disclosed and claimed inventive concepts is to illustrate the concepts of supplying/generating additional ATP for the muscle cell for the specific purpose of providing topical energy to muscles or treat any one of numerous possible symptoms associated with thereof. As demonstrated above, major metabolic pathways converge on the TCA cycle. Specifically: 1) The citric acid cycle is the third step in carbohydrate catabolism (the breakdown of sugars). Glycolysis breaks glucose (a six-carbon-molecule) down into pyruvate (a three-carbon molecule). Similarly, with the glycogen stores, catabolism of the complex carbohydrates into glucose leads to the same biochemical pathways. In eukaryotes, pyruvate moves into the mitochondria where it is converted into acetyl-CoA by decarboxylation and enters the citric acid cycle. 2) In protein catabolism, proteins are broken down by proteases into their constituent amino acids. The carbon backbone of these amino acids then becomes a source of energy by conversion to acetyl-CoA and entry into the citric acid cycle. And 3) in fat catabolism, triglycerides are hydrolyzed into three fatty acids and glycerol. In the liver the glycerol can be converted into glucose via dihydroxyacetone phosphate and glyceraldehyde-3-phosphate by way of gluconeogenesis, thus providing additional energy. More importantly, the fatty acids are broken down through β-oxidation, which results in acetyl-CoA used in the citric acid cycle. The total energy gained from the complete breakdown of one molecule of glucose by glycolysis, the citric acid cycle, and oxidative phosphorylation equals about 30 ATP molecules. However, glycogen, collagen and fatty acids (lipids) yield much greater amounts of ATP, the amount dependent on the amount and type of precursors used to generate the substrates needed to fuel the citric acid cycle; again, in general, the fatty acids yield the greatest amount of ATP. Other compounds may also contribute to energy production. Additional compounds may also amplify the process(es) of generating ATP within cells and may be included as potential agents in a topical application. The following compositions and the candidate compounds identified are earmarked as likely candidates to provide additional ATP in these topical application processes:

Pyruvate is a key by-product of the glycogenesis processes. The glycolytic pathway produces (from one glucose) two pyruvates, which in the presence of oxygen will be further metabolized in the citric acid cycle to produce NADH and FADH₂ for oxidative phosphorylation in the mitochondria. Normally, lactic acid will be low under these conditions. In the absence of oxygen (anaerobic), pyruvate must be converted to lactic acid, the only reaction that can regenerate NAD allowing further glycolysis. The production of lactic acid only under anaerobic conditions explains why the ratio of pyruvate/lactate is much less than 1 in anaerobic cells and much greater than one in aerobic cells. Under aerobic conditions pyruvate is converted to Acetyl-CoA by a series of enzymatic reactions. This process actually uses one molecule of ATP in the final step. The tradeoff is that there is more available Acetyl-CoA (to generate additional NADH/ATP). Acetyl-CoA then fuels the citric acid cycle which involves a series of enzymatic reactions to generate NADH, which in turn undergoes oxidative phosphorylation to form ATP. Some of the key intermediates in the citric acid cycle are (sequentially) citrate, succinate, fumarate and malate (see below). Although various entry and exist points occur for numerous cellular/biochemical pathways, the key outcome for cellular energy is ATP. Along these lines, an important co-enzyme is Coenzyme Q10 (CoQ10). CoQ10 is fat-soluble and is therefore localized in cellular membranes; it plays a unique role in the electron transport chain (ETC). In the inner mitochondrial membrane, electrons from NADH and succinate pass through the ETC to oxygen, which is then reduced to water. The transfer of electrons through the ETC results in the pumping of H⁺ across the membrane, creating a proton gradient across the membrane, which is used by ATP synthase (located on the membrane) to generate ATP. CoQ10 functions as an electron carrier from enzyme complexes I-III in this process. This is crucial in the process, since no other molecule can perform this function. Thus, CoQ10 functions in every cell, including muscle, to synthesize energy in the form of ATP.

With respect to the central role of acetyl-CoA in the shuttle of carbohydrate, fatty acid or protein metabolites into the citric acid cycle, it is interesting to note that pantothenic acid (vitamin B5) and cysteine (one of the amino acids) are the substrates used in the synthesis of Coenzyme A (the CoA in Acetyl-CoA). Yet another factor in Acetyl-CoA production is lipoic acid (aka thioctic acid or 6,8-dithiooctanoic acid; its base is liponate). Lipoic acid is an organosulfur compound derived from octanoic acid (one of the mid chain fatty acids). Lipoic acid is a coenzyme (in its protein bound form) for key metabolic enzymes essential for aerobic metabolism. Specifically, it is another key factor in the generation of Acetyl CoA, and subsequently additional ATP.

In particular the presently disclosed and claimed inventive concept(s) also relate(s) to methods to treat muscles using pyruvate (aka 2-oxopropanoate, 2-oxopropanoic acid, 2-oxypropanoic acid, acetylformic acid, alpha-keto acid, alpha-ketopropionic acid, calcium pyruvate, calcium pyruvate monohydrate). As noted above, pyruvate (C₃H₄O₃) is a key by-product of glycolysis from glucose (obtained from the blood or the breakdown of glycogen). Under anaerobic conditions pyruvate is converted (reduced) to lactate; under aerobic conditions it enters the citric acid cycle via conversion (via oxidative decarboxylation) to Acetyl CoA. Acetyl CoA then fuels the citric acid cycle which involves a series of enzymatic reactions to generate NADH, which in turn undergoes oxidative phosphorylation to form ATP. Providing supplemental topical pyruvate will provide a means to elevate ATP production. Preferably, compositions of the present invention contain a concentration of pyruvate (including all and any pyruvate compounds, analogs, or derivatives and pharmaceutically-acceptable hydrates and salts thereof that are suitable for being metabolized in a similar manner to endogenous pyruvate for the de novo synthesis of ATP) between 0.01% and 90% (w/v) of MFCA. Preferably, the pyruvate is between 1% and 15% (w/v) of the composition, inclusive of the endpoints. In other embodiments, the pyruvate is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one aspect, the pyruvate has a concentration of about 2.5% or 10% (w/v).

In particular, the presently disclosed and claimed inventive concept(s) also relates to method(s) to treat muscles using sodium lactate (NaC₃H₅O₃), the sodium salt of lactic acid. It is also called monosodium salt and lactic acid sodium salt. Sodium lactate is basically a liquid substance, but it can also be available in powder form. The liquid form is widely used for injection (e.g., Lactated Ringer's Solutions). Consideration for the use of lactate is based on the fact that in anaerobic glycolysis, NADH is reoxidized by conversion of pyruvate to lactate (without O₂), which leads to 2 ATP. Interestingly, heart muscle uses lactate as a major fuel source. Thus, even though the yield is low, it is important for energy for muscles (including skeletal muscles), especially with a low oxygen supply. Sodium lactate is also safe for topical use (e.g., it is widely used in cosmetics), but subject to a restriction on concentration of not more than 10% (w/v). Preferably, compositions of the present invention contain a concentration of between 1% and 90% (w/v) of lactate and includes all and any lactate compounds, analogs, or derivatives and pharmaceutically-acceptable hydrates and salts thereof that are suitable for being metabolized in a similar manner to endogenous lactate for the de novo synthesis of ATP. Preferably, the lactate is between 1% and 10% (w/v) of the composition, inclusive of the endpoints. In other embodiments, the lactate is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one aspect, the lactate has a concentration of about 2.5% or 7.5% (w/v).

In one particular composition, pyruvate and lactate may be formulated individually or in combination based on aerobic versus anaerobic conditions expected during muscle use.

In particular, the presently disclosed and claimed inventive concept(s) also relates to method(s) to treat muscles using Coenzyme Q10. Coenzyme Q10 (CoQ10; also known as ubiquinone) is a vitamin-related enzyme with an active role in the aerobic respiratory processes within cells (i.e., electron transport) for generating energy in the form of ATP. It is lipid (fat) soluble and is normally found in the membranes of all cells and in many of the cellular organelles (most notably mitochondria). The highest concentrations of CoQ10 are found in the most active muscles and organs such as heart, kidney and liver, followed closely by the long muscles, although all cells have and use it. CoQ10 is available from endogenous and exogenous sources. CoQ10 is safe as a paste, at lower doses in creams and lotions and at 2-8% in cosmetic products. It easily penetrates skin and there are no apparent side effects of topical application of CoQ10. CoQ10 may be formulated into a preferred embodiment apparent to those skilled in the art of compounding. (U Hoppe, J Bergemanna, W Diembecka, J Ennen, S Gohlaa, I Harris, J Jacob, J Kielholz, W Mei, D Pollet, D Schachtschabel, G Sauermanna, V Schreiner, F Stäb, F Steckel, Coenzyme Q10, a cutaneous antioxidant and energizer, BioFactors, (1999) 9:371-378; J Vinson, S I Anamandla, Comparative Topical Absorption and Antioxidant Effectiveness of Two Forms of Coenzyme Q10 after a Single Dose and after Long-Term Supplementation in the Skin of Young and Middle-Aged Subjects, IFSCC Magazine (2005)8:1-6). The potential use of CoQ10 in muscle physiology, and in particular muscular fatigue and cramp relief, is its impact on ATP generation in a site specific delivery via transdermal penetration. The predicted impact is to facilitate the de novo synthesis of ATP from existing glucose, glycogen, fatty acids and/or collagen stores, as well as from topically applied supplements as described above. It is also predicted that CoQ10 will have an additive and/or synergistic effect in providing additional ATP to a muscle.

CoQ10 compounds for use in the compositions and methods described herein include all and any CoQ10 compounds, analogs, or derivatives and pharmaceutically-acceptable hydrates and salts thereof that are suitable for being metabolized in a similar manner to endogenous CoQ10 for the de novo synthesis of ATP. Preferably, compositions of the present invention contain a concentration of CoQ10 wherein the amount of CoQ10 delivered to the affected muscle cells after topical application is greater than that found endogenously. A composition of the present invention may contain between 0.01% and 90% by weight (w/v) of CoQ10. Preferably, the CoQ10 is between 1% and 20% (w/v) of the composition, inclusive of the endpoints. In other embodiments, the CoQ10 is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one particular embodiment, the CoQ10 has a concentration between 1% and 15% (w/v), for example, a concentration of about 1.3% or 13.5% (w/v).

In particular, the presently disclosed and claimed inventive concept(s) also relates to method(s) to treat muscles using lipoic acid. Lipoic acid (aka thioctic acid or 6,8-dithiooctanoic acid; its base is liponate) is an organosulfur compound derived from octanoic acid (one of the MCFAs; see above). Lipoic acid is an essential coenzyme (in its protein bound form) for key metabolic enzymes for aerobic metabolism and ATP energy production. The amount of free lipoic acid present is very low, due to the fact that it is normally bound to proteins (a form of sequestration); the amount of lipoic acid bound to plasma proteins is reported to be 12.3-43.1 ng/ml and to cellular proteins at 1.4-38.2 ng/ml. It is readily soluble in water and fat. It is considered safer than topically applied vitamin C or E and is used topically at 0.2%-5.0% (w/v; typically as anti-aging creams due to its antioxidant properties). (L Packer, S M Patel, eds., Lipoic acid: energy production, antioxidant activity and health effects, CRC Press (2008), Boca Raton, Fla.). The predicted impact of supplemental lipoic acid is to facilitate the de novo synthesis of ATP from existing glucose, glycogen, fatty acids and collagen, as well as from topically applied supplements as described above. It is also predicted that lipoic acid used alone or in conjunction with CoQ10 will have an additive and/or synergistic effect in providing additional ATP to a muscle.

Lipoic acid for use in the compositions and methods described herein include all and any lipoic acids, analogs, or derivatives and pharmaceutically-acceptable hydrates and salts thereof that are suitable for being metabolized in a similar manner to endogenous lipoic acid for the de novo synthesis of ATP. Preferably, compositions of the present invention contain a concentration of lipoic acid wherein the amount of lipoic acid delivered to the affected muscle cells after topical application is greater than that found endogenously. A composition of the present invention may contain between 0.01% and 90% by weight (w/v) of lipoic acid. Preferably, the lipoic acid is between 1% and 10% (w/v) of the composition, inclusive of the endpoints. In other embodiments, the lipoic acid is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90%, by weight (w/v) of the composition. In one particular embodiment, the lipoic acid has a concentration of between 1% and 5% (w/v); e.g., a concentration of about 1.3% (w/v) of the composition.

In another embodiment, a composition of the present invention may contain a combination of lipoic acid and CoQ10, wherein the combination is present between 0.1% and 90% by weight (w/v) of the composition, inclusive of the endpoints. In one particular embodiment, the combination of lipoic acid and CoQ10 has a concentration of between 2% and 15% (w/v); e.g., of about 2.6% or about 14.8% (w/v) of the composition; considerations of the formulations of individual components with be considered in the combination composition.

In particular, the presently disclosed and claimed inventive concept(s) also relates to method(s) to treat muscles using numerous other precursors, cofactors, substrates, metabolites, etc., in the generation of ATP. The following are presented as examples that are not intended to be limiting:

In one aspect, the present invention features a method wherein the composition contains one or a combination of two or three of pantothenic acid (vitamin B5), adenosine diphosphate (ADP) and cysteine, which are necessary for the biosynthesis of Acetyl-Coenzyme A (Acetyl-CoA), as a means to internally generate additional Acetyl-CoA. The Coenzyme A moiety is actually a Pantothenic Acid molecule attached to an ADP molecule (via the cysteine). (G F Jr Combs, The vitamins: Fundamental Aspects in Nutrition and Health. (3rd ed), Elsevier Academic Press (2008) Ithaca, N.Y.; P R Trumbo, Pantothenic Acid, In M E Shils, M Shike, A C Ross, et al. Modern Nutrition in Health and Disease (10^{th} ed.), Lippincott Williams & Wilkins (2006) Philadelphia, Pa.). As described above, Acetyl-CoA is an important factor in shuttling metabolites of carbohydrates, proteins and fatty acids into the citric acid cycle leading to the subsequent generation of ATP. Pantothenic acid for use in the compositions and methods described herein include all and any lipoic acids, analogs, or derivatives and pharmaceutically-acceptable hydrates and salts thereof that are suitable for being metabolized in a similar manner to endogenous pantothenic acid for the de novo synthesis of ATP. Preferably, compositions of the present invention contain a concentration of pantothenic acid wherein the amount of pantothenic acid delivered to the affected muscle cells after topical application is greater than that found endogenously. A composition of the present invention may contain between 0.01% and 90% (w/v) of pantothenic acid. Preferably, the pantothenic acid is between 1% and 20% (w/v) of the composition, inclusive of the endpoints. In other embodiments, the pantothenic acid is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one particular embodiment, the pantothenic acid has a concentration of between 5% and 15% (w/v); e.g., a concentration of about 10% (w/v) of the composition.

In one aspect, the present invention features a method wherein the composition contains citrate, fumarate, malate or succinate, either individually or in one of more combinations thereof. These four compounds play important intermediary roles in the citric acid cycle and thus the generation of cellular energy. Although their use alone may be beneficial in promoting the citric acid cycle in a scenario with limiting intermediates, the proposed use of these intermediates is in conjunction with one or more energy sources which will boost the utilization of metabolites entering into the citric acid cycle. These compounds and their carboxylic acids are not considered dangerous in light of the fact that they are naturally found in all cells, although at high concentrations some may be a skin irritant. A composition of the present invention may contain between 0.01% and 90% by weight (w/v) of one or more (individually or in combination(s) of two or more) of citrate, fumarate, malate or succinate. Preferably, the compound is between 1% and 20% (w/v) of the composition, inclusive of the endpoints. In other embodiments, these compounds individually or in combination is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one particular embodiment, these compounds, individually or in one or more combinations, has a concentration of between 1% and 10% (w/v); e.g., a concentration of about 5% (w/v) of the composition.

In one aspect, the present invention features a method wherein the composition contains glutamine. This amino acid is converted though several enzymatic steps to oxaloacetate, which is also a key intermediate in the citric acid cycle. Oxaloacetate is also an intermediate in gluconeogenesis and the formation of citrate. A composition of the present invention may contain glutamine between 0.01% and 90% (w/v). Preferably, glutamine is between 0.01% and 20% (w/v) of the composition, inclusive of the endpoints. In other embodiments, glutamine is at least 0.01%, 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one particular embodiment of glutamine has a concentration of between 0.01% and 10% (w/v); e.g., a concentration of about 0.1% (w/v) of the composition.

In one aspect, the present invention features a method wherein the composition contains the addition of L-methionine, glycine, and arginine, which are amino acid precursors necessary for the biosynthesis of creatine (see above). As such the use of supplemented L-methionine, glycine and arginine may impact the regenerative properties of cells, converting ADP to ATP, by providing precursors for the de novo intracellular synthesis of creatine. (M Wyss, R Kaddurah-Daouk, Creatine and Creatinine Metabolism, Physio Rev (2000) 80:1108-1213; A M Persky, G A Brazeau, Clinical Pharmacology of the Dietary Supplement Creatine Monohydrate, Pharmacol Rev (2001) 53:162-176). A composition of the present invention may contain between 0.01% and 90% (w/v) of an equimolar ratio of L-methionine, glycine, and arginine. Preferably, a composition, containing equimolar ratios of these amino acids, contain between 1% and 20% (w/v) of the composition, inclusive of the endpoints. In other embodiments, these compounds (in equimolar ratios) are at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one particular embodiment, these compounds have a concentration of between 0.1% and 10% (w/v); e.g., a concentration of about 1.5% (w/v) of the composition.

In one aspect, the present invention features a method wherein the composition contains the addition of Nicotinamide Adenine Dinucleotide (NADH), which is a key component (coenzyme) linking the citric acid cycle with oxidative phosphorylation in the generation of ATP from ADP. NADH yields 3 ATP per molecule. As such the use of NADH as a supplement in a topical application is predicted to increase ATP production. A composition of the present invention may contain NADH between 0.01% and 90% by weight (w/v). Preferably, NADH is between 0.01% and 20% (w/v) of the composition, inclusive of the endpoints. In other embodiments, NADH is at least 0.01%, 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one particular embodiment of NADH has a concentration of between 0.01% and 10% (w/v); e.g., a concentration of about 0.1% (w/v) of the composition.

In one aspect, the present invention features a method wherein the composition contains the addition of Flavin Adenine Dinucleotide (FAD as the reduced form and/or FADH₂ as the oxidized form). The primary biochemical role of FADH₂ is to carry high-energy electrons used for oxidative phosphorylation of ADP to form ATP. FADH₂ yields 2 ATP per molecule. As such the use of FADH₂ is predicted to increase ATP production. A composition of the present invention may contain FADH₂ between 0.01% and 90% by weight (w/v). Preferably, FADH₂ is between 0.01% and 20% (w/v) of the composition, inclusive of the endpoints. In other embodiments, FADH₂ is at least 0.01%, 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one particular embodiment of FADH₂ has a concentration of between 0.01% and 10% (w/v); e.g., a concentration of about 0.1% (w/v) of the composition.

In one aspect, the present invention features a method wherein the composition contains the addition of ketone bodies. Ketone bodies, (acetoacetate and β-hydroxybutyrate, the two functional ketone bodies), are formed from fatty acid oxidation and the catabolism of some amino acids. These ketone bodies can be oxidized as fuels to form pyruvate and subsequently Acetyl CoA, which enters the citric acid cycle. In low glucose situations, they can provide an alternative energy source, producing high levels of ATP. They are normally present at relative high amounts (1% w/v) in equimolar ratios in blood, but may increase considerably in ketosis. Ketone bodies are aqueous-soluble, do not require facilitated transport and easily penetrate into cells for immediate use for cellular energy. A composition of the present invention may contain one or a mixture (in one of multiple ratios) of acetoacetate and β-hydroxybutyrate between 0.1% and 90% (w/v). Preferably, the concentration of one or a combination of the two ketone bodies is between 0.1% and 20% (w/v) of the composition, inclusive of the endpoints. In other embodiments, the concentration is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one particular embodiment of acetoacetate and/or β-hydroxybutyrate has a concentration of between 0.1% and 10% (w/v); e.g., a concentration of about 2.5% (w/v) of the composition.

In one aspect, the present invention features a method wherein the composition contains potassium (K). Along these lines, an electrolyte imbalance has been commonly implicated as a causative factor for cramps (one application of the currently disclosed and claimed inventions), although current research does not directly support the anecdotal data (see above). Nevertheless, a considerable amount of effort has been directed at maintaining several electrolytes of interest via oral routes; specifically sodium, calcium, chloride, magnesium and potassium. All five play key roles in cellular physiology and are highly regulated within all cells (see below). Since the cellular membrane is impermeable to these electrolytes, highly specialized protein carriers and transporters are imbedded in the cellular membrane to maintain the necessary concentrations for cellular function. This may involve excluding (pumping out) ions or actively bringing into the cell the necessary ions. The concentrations of these electrolytes inside and outside the cell are listed below. Note that with respect to muscular activity, calcium plays a key regulatory role in neuronal stimuli for muscle contraction, yet in the generation of ATP for actual contraction, K is key.

| | Intracellular Conc. | Extracellular Conc. |
|---|---|---|
| Component | (μM) | (μM) |
| Na | 5-15 | 145 |
| Cl | 5-15 | 110 |
| K | 140 | 5 |
| Mg | 0.5 | 1-2 |
| Ca | 0.0001 | 1-2 |

In general the cell goes to considerable lengths to exclude salt (NaCl) and calcium and to a lesser extent magnesium, and to concentrate intracellular potassium. Intuitively, of the 4 cations listed, potassium is the only one with major constraints in terms of electrolyte concentration limitations in relation to potential imbalances within versus outside the cell (i.e., more is needed than is readily available). One of the objectives of a large family of sports drinks is to reconstitute these electrolytes in general after dehydration from fluid lost from the interstitial tissues during intense exercise. However, oral drinks are limited in terms of delivery to the specific sites of need (i.e., dilutional effects, temporal issues, first pass issues through the liver, and potential side effects, e.g., with the kidney). In light of the necessity of K, the inclusion of K in the formulation of topical treatments of muscles is important, yet relatively straightforward, by using a potassium salt, rather than sodium or other salt, of a compound(s) of interest(s). This is intended to be a supplemental addition, rather than a primary component in formulations for muscle treatment. Concentrations of K in the composition are between 0.1 mM and 100 mM.

In one aspect, a composition of the present invention may comprise carnosine or one or more of precursors of carnosine. Although carnosine is not directly involved in ATP production, there is strong evidence for its role in mitigating adverse effects of muscle contraction and more specifically the impact of prolonged muscle contractions (e.g., buffering pH effects of lactic acid), while improving muscle mass and recovery post exercise. Carnosine is a dipeptide of beta-alanine and histidine found predominately in muscle. The rate limiting step in its production is the availability of beta-alanine. As noted, carnosine mitigates some of the adverse effects of exercise and as such as been a candidate for study in exercise physiology. However, the oral use of carnosine as a supplement is limited due to GI catabolism issues. As such, current oral supplementation studies have focused on beta-alanine and have demonstrated the efficacy of using beta-alanine to enhance the generation of carnosine in muscle. Topical application of either carnosine and/or one (beta-alanine) or more (beta-alanine plus histidine) precursors provides an alternative delivery directly to the muscles. A composition of the present invention may contain one or a mixture (in one of multiple ratios) of carnotine, beta-alanine and histidine between 0.1% and 90% (w/v). Preferably, the concentration of one or a combination of these compounds is between 0.1% and 20% (w/v) of the composition, inclusive of the endpoints. In other embodiments, the concentration is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90% (w/v) of the composition. In one particular embodiment of carnosine or beta-alanine has a concentration of between 0.1% and 10% (w/v); e.g., a concentration of about 2.5% (w/v) of the composition. Compounds for use in formulation include the numerous commercially available forms of carnosine, beta-alanine and histidine or derivatives thereof suitable for the purposes herein described.

In one aspect, a composition of the present invention may comprise any combination of two or more of claimed inventive concepts and processes. Their impact is intended to increase muscle performance, to improve the efficacy in the treatment muscles, and to improve methods for the prevention of various muscle conditions, than a composition comprising any of the listed components alone. It should be noted that an experimental sport's study has been conducted, demonstrating the efficacy of this approach (see below).

In particular, the presently disclosed and claimed inventive concept relates to methods to treat muscles and may also include one or more analgesics to treat pain and inflammation often associated with a muscle condition (e.g., muscle fatigue or cramps). An analgesic is defined as any member of the group of drugs used to achieve relief from pain; some are also effective as anti-inflammatory agents. Commonly known as painkillers, analgesic drugs act in various ways on the peripheral and central nervous systems. They include, but are not limited to, paracetamol (known in the US as acetaminophen) and the non-steroidal anti-inflammatory drugs (NSAID) such as the salicylates (e.g., aspirin or trolamine salicylate), ibuprofen and naproxen. For prescription purposes, the opioid drugs (e.g., the morphine and opium related compounds) as analgesics are also included. (C K S Ong, P Lirk, C H Tan, R A Seymour, An Evidence-Based Update on Nonsteroidal Anti-Inflammatory Drugs, Clin Med Res (2007) 5:19-34; Quantitative systematic review of topically applied non-steroidal anti-inflammatory drugs, R A Moore, M R Tramèr, D Carroll, P J Wiffen, H J McQuay, BMJ (1998) 316:333-338). One particular analgesic is trolamine salicylate.

A composition of the present invention may contain between 0.01% and 25% by weight (w/v) of analgesic. Preferably, the analgesic is between 0.5% and 15% by weight (w/v) of the composition, inclusive of the endpoints. In other embodiments, the analgesic is at least 0.01%, 0.02%, 0.03%, 0.04%, 0.05%, 0.06%, 0.07%, 0.08%, 0.09%, 0.1%, 0.25%, 0.5%, 0.75%, 1%, 2%, 3%, 4%, 5%, 6%, 7%, 8%, 9%, 10%, 12.5%, or 15% by weight (w/v) of the composition. In one particular embodiment, the analgesic (i.e., trolamine salicylate) has a concentration of between 5% and 10% (w/v) of the composition, for example, a concentration of about 6.7%.

In another embodiment, the present invention may also contain menthol, methyl salicylate (wintergreens), eucalyptus oil, capsaicin as alternative or additional topical analgesics to mitigate pain and/or to provide heat or cooling effects to the treated site.

In particular, the presently disclosed and claimed inventive concept relates to methods to treat muscles and may also include one or more compounds to affect penetration of the compounds through the skin after topical application of the formula. A topical application implies an effective composition topically applied to the skin in the area of the body which is targeted for treatment or is experiencing discomfort from muscle fatigue, a cramp or muscle condition (e.g., atrophy or a myopathy). A topical application may be in the form of a solution, emulsion, lotion, gel, jelly, ointment, cream, paste or plaster, spray or aerosol, roll-on, semi-solid (stick), patch, etc. The “skin” normally presents a formidable barrier that minimizes the egress of physiological fluids while preventing the ingress of toxic chemicals and potential pathogens. Penetration enhancer(s) facilitate the percutaneous absorption of compounds of interests from the skin surface into the stratum corneum (approximately 10 mm and considered the most difficult to transit) and subsequently through the stratum corneum and epidermis, through the dermis and into the underlying tissues. Their effect(s) are to safely and temporarily diminish the impermeability of skin. This is generally achieved through transepidermal absorption or transfollicular (shunt pathway) absorption. Non-limiting examples of possible penetration enhancers are: alcohols (methanol and various alcohol derivatives; e.g., lauryl alcohol), glycols (e.g., propylene glycol), glycerides (e.g., glycerin or caprylic acid triglyceride), various terpenes (and their derivatives, the terpenoids), essential oils (e.g., eucalyptus, peppermint, turpentine oil, etc.), fatty acids and their esters (e.g., octanoic acid, decanoic acid, lauric acid, oleic acid, etc.), nonionic surfactants (e.g., bile salts), pyrrolidones and their derivatives (e.g., N-methyl-2-pyrrolidone), azones (e.g., laurocapram), phospholipids and derivatives thereof (e.g., phosphtidyl glycerol derivatives), cyclodextran complexes, and sulfoxides (e.g., dimethyl sulfoxide; DMSO), to name a few. Reviews of the field of penetration enhancers which elucidate the state of the art are herein cited. (V R Sinha, M P Kaur, Permeation Enhancers for Transdermal Drug Delivery, Drug Dev Industrial Pharmacy, (2000), 26:1131-1140; M R Prausnitz, R Langer, Transdermal drug delivery, Nat Biotechnol, (2008) 26:1261-1268; D W Osborn, J J Henke, Skin Penetration Enhancers Cited in the Technical Literature, Pharmacology Tech (1997); H-Y Thong, H Zhai, H I Maibach, Percutaneous Penetration Enhancers: An Overview, Skin Pharmacol Physiol (2007) 20:272-282)

A composition of the present invention may contain between 0.01% and 90% by weight (w/v) of a penetration enhancer compound. Preferably, the penetration enhancer compound is between 0.1% and 20% by weight (w/v) of the composition. In other embodiments, the penetration enhancer compound is at least 0.1%, 0.25%, 0.5%, 0.75%, 1%, 2%, 3%, 4%, 5%, 6%, 7%, 8%, 9%, 10%, 12.5%, 15%, 17.5%, 20%, 25%, 30%, 40%, 50%, 60%, 70%, 80%, or 90%, by weight (w/v) of the composition.

In particular, the presently disclosed and claimed inventive concept(s) relates to methods to treat muscles and may also include one or more essential oil(s). Essential oils used herein refer to natural oils extracted by one of various methods known to those skilled in the art from bark, flowers, fruits, herbs, seeds, trees and other plants. They tend to be aromatic and volatile and are insoluble in water, but rather soluble in various natural or synthetic organic solvents such as fatty acid, lipids and oils (e.g., vegetable oil), alcohol, and ether. Examples of essential oils which may be used in the context of the presently disclosed and claimed inventive concept(s) include, but are not limited to: agarwood oil, almond oil, anise oil, arnica (Montana) oil, balsam oil, basil, bergamot oil, black pepper oil, buchu oil, camphor oil (white or yellow), cannabis oil, cardoman oil, cassia oil, cedar oil, cedar leaf oil, celery oil, chamomile oil, cinnamon oil, sage oil, clary sage oil, clove oil, clove leaf oil, coriander oil, cumin, cypress oil, evening primrose oil, eucalyptus oil, fennel oil, fistree oil, frankincense oil, joram oil, geranium oil, ginger oil, grapefruit oil, guava oil, ho oil, hops oil, hyssop oil, jasmine oil, jojoba oil, juniper oil, lavender oil, lemon oil, lemongrass oil, lime oil, macadamia nut oil, mandarin oil, manuka oil, marjoram oil, myrrh oil, melaleuca oil, menthol, neroli oil, neem oil, nutmeg oil, orange oil (various oranges, to include blood orange), palmarosa oil, patchouli oil, pepper oil, peppermint, petitgrain oil, pimento berries oil, pine seed oil, pine needle oil, ravensara oil, rose otto oil, rosewood oil, rosemary oil, sandalwood oil, sassafras oil, sea fennel oil, sesame oil, Spanish rosemary oil, Spanish sage oil, spearmint oil, spikenard oil, tarragon, tea tree oil, thyme oil, tsuga rose oil, thyme, turpentine oil, valerian oil, vetiver oil, walnut oil, whitepine oil, wintergreen oil, or ylang ylang oil.

A composition of the present invention may contain between 0.01% and 20% by volume (v/v) of an essential oil(s). Preferably, the essential oil(s) is between 0.1% and 5% by volume (v/v) of the composition. In other embodiments, the essential oil(s) is at least 0.01%, 0.02%, 0.03%, 0.04%, 0.05%, 0.06%, 0.07%, 0.08%, 0.09%, 0.1%, 0.25%, 0.5%, 0.75%, 1%, 2%, 3%, 4%, 5%, 6%, 7%, 8%, 9%, 10%, 12.5%, 15%, 17.5%, or 20% (v/v) of the composition. In one particular embodiment, the essential oil(s) have a concentration of between 0.1% to 5% (v/v) of the composition, for example, a concentration of about 1.5%, 1.7%, or 1.8% (v/v).

Optimization of specific formulations of the compositions as described herein can be performed by one ordinarily skilled in the art and the selection of the various compositions and their delivery will clearly be influenced by professional(s) in the relevant field. The method of delivery or administration can be through topical application. To affect the delivery of the topical application, the formulation may be in the form of a solution, emulsion, lotion, gel, jelly, ointment, cream, paste or plaster, spray or aerosol, roll-on, semi-solid (stick), transdermal patch, etc. For example, the transdermal patch is adhered to the surface of the skin and contains a reservoir or layer containing compositions of the present invention. This embodiment will be commensurate with the desired/necessary delivery for the particular demographic which will utilize the product. The composition(s) may also contain stabilizers, preservatives, buffers, antioxidants, an acceptable carrier or other formulation. (Remington: The Science and Practice of Pharmacy, 21st edition, Lippincott Williams & Wilkins, Philadelphia, Pa. (2005))

The combination and effective amounts of active compounds, such as Acetyl-CoA), adenosine triphosphate (ATP), citrate, Coenzyme Q10 (CoQ10), fumarate, carnosine, collagen (as examples, hydrolyzed collagen, various peptides, etc.), cysteine, beta-alanine (or other amino acids), glucose, glycogen, lactate/lactic acid, lipoic acid/liponate, malate, Medium (Mid) Chain Fatty Acid (MCFA), nicotinamide adenine dinucleotide (NADH), pantothenic acid (vitamin B5), pyruvate, and/or succinate may be dependent upon the specific applications (e.g., medical professionals, physical therapists, certified athletic trainers, massage therapists, etc.), type of muscle and/or muscle activity and muscular disorder (e.g., type or severity of muscle fatigue, nocturnal or athletic cramp, therapy modality or myopathy), etc.

Suitable dosages for administration can be determined by a clinical or health-care professional, physical therapist, or athletic trainer. Dosages may be dependent upon the type of muscle treated, the muscle condition in treatment, the severity of muscle fatigue, the type of cramp, the muscle treatment/therapy used, the myopathy under treatment, the athletic activity, etc. Preferably, the compositions are administered at dosages and at a dosing regimen sufficient to yield a desired therapeutic response (e.g., alleviation or reduction of a muscle cramp, treatment a muscle symptom such as fatigue or a strain, cramp relief or prevention, improvement of a myopathy, etc.) without undue adverse side effects (such as toxicity, irritation, or allergic response) commensurate with a reasonable benefit/risk ratio when used in the manner of this invention. Compositions of the present invention may be administered once, or administered repeatedly, or at repeated intervals.

As used herein, “subject in need thereof” or “subjects in need thereof” refers to any individual who is suffering, has suffered, or is at risk of suffering from a muscle symptom. Examples of exemplary subjects include, but are not limited to, individuals that experience cramps frequently, regularly, or sporadically, elderly individuals that experience nocturnal cramps, individuals that experience nocturnal cramps, women that suffer from menstrual cramps, amputees, various cramp-prone athletes (e.g., tennis players, marathon runners, swimmers, football players, and individuals that participate in rigorous physical activities and exercise), treatments post exercise in athletes experiencing fatigue, exhaustion, or strains, etc.

As used herein, “treating” or “treat” describes the management and care of a subject for the purpose of eliminating, relieving, alleviating, or reducing the severity of a muscle condition or symptom, such as fatigue or a cramp or any symptom associated with a muscle condition or symptom, and includes the administration of a composition of the present invention. The elimination, relief, alleviation, or reduction in severity of the muscle symptom associated can occur immediately or some time after administration of the composition. Reduction in severity of the muscle condition includes reduction in the number or duration of the muscle symptom.

The term “preventing” or “prevent” as used herein includes either preventing the onset of muscle condition (e.g., fatigue or cramp) altogether, preventing or slowing the onset of a muscle condition in individuals or subjects at risk, relieving a muscle symptom, or improving a muscle symptom. This includes prophylactic treatment of those at risk. In some aspects, the compositions of the present invention are administered prior to engagement in activities that may induce a cramp or if the subject is prone to or suspects a cramp will occur; e.g., before a competitive game or match, before exercising, or before sleeping. In other aspects, treatment with the desired composition may be included in certain therapies; e.g., rehabilitation after injury, reconditioning after extended bed rest or a broken bone requiring immobilization with a sling or cast leading to atrophy, physical therapy for certain myopathies, etc.

As used herein, the term “alleviate” or “ameliorate” is meant to describe a process by which the severity of a symptom of, or associated with, a muscle symptom is decreased. Importantly, a symptom can be alleviated without being eliminated. In one embodiment, the administration of compositions of the invention leads to the elimination of a symptom; however, elimination is not required. Therapeutically effective dosages are expected to decrease the severity of a symptom.

As used herein, the term “symptom” is defined as an indication of disease, illness, injury, or that something is not right in the body. Symptoms are felt or noticed by the individual experiencing the symptom, but may not easily be noticed by others. Others are defined by non-health-care professionals. Symptoms of a muscle include, but are not limited to, muscle spasms, muscle pain, pain at the site of the muscle, muscle rigor or stiffness, and muscle soreness or weakness. This may include ensuing symptom(s) after the primary symptom.

As used herein, the term “about”, in reference to ranges and concentrations, means within 0.1% or 1% or 2% of the indicated concentration.

One topical application is to relieve cramps by providing additional ATP at the site of the cramp. The premise of such treatment is that relief of the rigor/cramped/contracted/stiff/spasmed muscle will be provided 1) from additional ATP (immediately available from free ATP topically applied), 2) from additional ATP generated as newly synthesized ATP, e.g., via CoQ10, lipoic acid, citrate, fumarate, malate or succinate, etc., as topically applied cofactors, coenzymes, substrates, etc., in the citric acid cycle to generate ATP, 3) from the de novo synthesis of intracellular factors to boost regeneration or generation of ATP (e.g., addition of amino acid precursors for creatine and/or pantothenic acid (vitamin B5) and cysteine for the biosynthesis of Acetyl-CoA, 4) from utilization of glucose, glycogen, MCFA or collagen as topically applied metabolites for generating additional ATP, and/or supplemental factors to assist or mitigate adverse effects in muscle physiology.

Muscle cramps are sudden, involuntary contractions or spasms in one or more muscles. They often occur after exercise or at night, lasting a few seconds to several minutes or longer. Examples of types of muscle cramps that can be treated using the methods and compositions disclosed herein include, but are not limited to, muscle spasm, “Charlie horse”, nocturnal or night-time cramps, menstrual cramps, heat cramp, writer's cramp, runner's cramp, swimmer's cramp, or a cramp experienced by an amputee. Symptoms associated with muscle cramps include muscle spasms, muscle rigor or stiffness, muscle pain (pain at the site of the muscle cramp), muscle soreness, muscle weakness or fatigue, and reoccurrence after the cessation of the cramp. The methods and compositions disclosed herein can be used to treat, alleviate, or prevent a muscle cramp. For example, the methods and compositions disclosed herein are used to reduce the severity of a cramp, reduce the number of cramps, reduce the duration of the cramp, or prevent the occurrence or reoccurrence of a cramp. Further, the methods and compositions disclosed herein can be used to alleviate a symptom of, or associated with, a muscle cramp.

In particular, the presently disclosed and claimed inventive concept(s) relates to methods to treat cramps and also includes one or more agents in one embodiment apparent to those skilled in the art of compounding.

Preferably, the compositions of the present invention are administered or applied topically to the site or surrounding area of the muscle cramp. In some embodiments, alleviation of the cramp or a symptom associated with the cramp, such as pain, muscle spasms, or muscle rigor, occurs immediately after administration. In other embodiments, the alleviation of the cramp or symptom of the cramp occurs within 1 to 30 minutes after administration. Treatment of the cramp or a symptom thereof can be effective within 3 to 10 minutes, 3 to 15 minutes, or 3 to 20 minutes of topical administration. In a further embodiment, a muscle cramp or a symptom of a muscle cramp is prevented for 1 to 24 hours after administration. For example, a muscle cramp or a symptom thereof can be prevented for 1 to 6 hours, 1 to 12 hours, or 1 to 18 hours after administration.

Examples are provided below. However, the presently disclosed and claimed inventive concept(s) is to be understood to not be limited in its application to the specific experimentation, results and laboratory procedures presented herein. Rather, the Examples are simply provided as one of various embodiments and are meant to be exemplary, not exhaustive.

### EXAMPLES

### Example 1: Formulations Containing Creatine; Proof of Concept

Initial studies utilized the following formulations (Formulas 1 and 2) to examine the efficacy of regenerating ATP using creatine in topical applications for cramp relief.

TABLE 1
Formulation 1

| Ingredient | Amount |
|---|---|
| Creatine monohydrate | 256 g |
| Chamomile Oil | 22.4 ml |
| Lavender Oil | 22.4 ml |
| Eucalyptus Oil | 12.5 ml |
| Clary Sage Oil | 6.8 ml |
| Lotion Base (carrier) | 3785 ml |

TABLE 2
Formulation 2

| Ingredient | Amount |
|---|---|
| Trolamine salicylate | 256 g |
| Creatine monohydrate | 256 g |
| Chamomile Oil | 25.0 ml |
| Lavender Oil | 25.0 ml |
| Eucalyptus Oil | 12.5 ml |
| Clary Sage Oil | 6.8 ml |
| Lotion Base (carrier) | QS to 3785 ml |

An open label (blind) study using Formulation 1 was conducted at seven athletic programs over a 30 day period in September-October, 2012, by certified athletic trainers under the supervision of an orthopedic surgeon. Athletes suffering from muscle cramps during either football practice sessions or games were treated by topical application of the composition to the affected limb or area. Each applied dose was sufficient to provide approximately 0.5 to 2.5 g of the creatine compound to the affected limb or area. The primary endpoint was cessation of exercise-related muscular cramps, with a secondary endpoint of non-recurrence for the remainder of sport event or practice period. All facilities provided fluid and electrolyte replacement ad libitum for the athletes before and during activities.

Of the 54 athletes who experienced cramps, 53 responded positively within 3-10 minutes from the onset of the cramp and 100% of the 53 were able to immediately resume athletic activities. The one failure to relieve a cramp in the one non-responder was attributable to profuse sweating and the inability to absorb the compound. Equally important, approximately 15% of the athletes were identified as “chronic crampers”, i.e., every athletic event precipitated a “cramp” event. Pretreatment before exercise with the disclosed formula prevented subsequent “cramp” events. It should be noted that prior to use of Formulation 1, treatment of cramps consisted of massage, electrolytes, hydration, and various remedies (e.g., homeopathic remedies of bananas, pickle juice, mustard consumption, etc.). Typical responses from former methods were no cramp relief, prolonged cramps, reoccurrence and/or the inability to continue physical exercise.

These results clearly demonstrated the effectiveness of a composition containing a creatine compound and at least one essential oil (i.e., Formulation 1) in the treatment and cessation of episodes of muscle cramps, in the inhibition of a reoccurrence of the muscle cramp and in the prevention of muscle cramping by prophylactic treatment with the composition. Treatment of an episode of muscle cramping was generally effective within 3 to 10 minutes of topical application of Formulation 1. Inhibition of reoccurrence of the muscle cramp after the initial event (episode) was effective for up to at least 1 hour and up to 24 hours.

Formulation 2 was compounded as with Formulation 1, but with an analgesic to conform to FDA standards for an over the counter (OTC) product (External analgesic drug products for over-the-counter human usage. Department of Health and Human Services, Food and Drug Administration. 21 CFR 348. Federal Registry (1987); 48(27):5852-5869). Similar results were obtained (i.e., >98% effective).

### Example 2: Formulations not Containing Creatine; Proof of Concept of ATP Supplementation or Generation

Subsequently, studies addressed utilizing the following compositions (Formulas 3-5) to examine the efficacy of either supplementing ATP or generating ATP without using creatine in topical applications. Although specific formulas have been disclosed, the inference is that in any of the these and ensuing formulations, the amounts and compound(s) may be optimized depending on the subject, muscle type, type of muscle symptom, and/or requirement(s) of the health professional, athletic trainer, physical therapist, etc. In any of these formulations, any of the essential oils, analgesics, or pharmaceutically-acceptable carrier(s) may be substituted with other essential oils, analgesics or pharmaceutically-acceptable carrier(s) disclosed herein by accomplished individuals knowledgeable of the art of compounding.

TABLE 3
Formulation 3

| Ingredient | Amount |
|---|---|
| Trolamine salicylate | 256 g |
| ATP | 50 g |
| CoQ10 | 50 g |
| Coconut oil | 512 g |
| Arnica Oil | 25 ml |
| Blood Orange Oil | 5 ml |
| Chamomile Oil | 25 ml |
| Lotion Base (carrier) | QS to 3785 ml |

TABLE 4
Formulation 4

| Ingredient | Amount |
|---|---|
| Trolamine salicylate | 256 g |
| ATP | 5 g |
| CoQ10 | 512 g |
| Lipoic Acid | 50 g |
| C8 | 256 g |
| C12 | 256 g |
| Arnica Oil | 25 ml |
| Blood Orange Oil | 5 ml |
| Chamomile Oil | 25 ml |
| Lotion Base (carrier) | QS to 3785 ml |

TABLE 5
Formulation 5

| Ingredient | Amount |
|---|---|
| C8 | 128 g |
| C12 | 128 g |
| Arnica Oil | 75 ml |
| Blood Orange | 5 ml |
| Chamomile Oil | 25 ml |
| Lotion Base | QS to 3785 ml |

A study of the effectiveness of the disclosed formulas was based on the following. Open label studies using the desired compositions as described herein have been performed to substantiate the efficacy for treating muscle cramps, alleviating a symptom of muscle cramps, and preventing reoccurrence of muscle cramps. Preferred subjects for these studies included individuals that suffer from regular or sporadic nocturnal cramps, women that suffer from menstrual cramps, amputees, and various cramp-prone athletes; e.g., tennis players, marathon runners, swimmers, football players, and individuals that participate in rigorous physical activities and exercising (mountain biking, triathlons, etc.). Studies involving high school, collegiate or professional athletic programs were/are conducted or supervised by physicians, designated health care professionals and certified athletic trainers. Results from nocturnal cramps or other noted cramps were self-reported.

In supervised athletic programs, individuals suffering from muscle cramps were/are treated by topical application of the designated composition to the affected limb or area, by a health professional or trainer. Each applied dose was/will be sufficient to provide a pharmaceutical dose of the compound(s) of interest. The primary endpoint was/is cessation of muscular cramps, with a secondary endpoint of non-recurrence for a predetermined period of time. Athletes were provided fluid and electrolyte replacement ad libitum before and during activities. Data on length of time to cessation of the cramp, degree of associated pain, frequency of cramps during a predetermined amount of time (for example, during training or competition), efficacy in minimizing reoccurrence, etc., were/are collected and compiled for reference.

To date, >250 individuals have successfully used Formulas 2-5 for nocturnal cramp relief and >100 athletes in organized sports programs, women with menstrual cramps, and other athletes have successfully used Formulas 2-5 for the prevention or relief of exercise or menstrual induced cramps. Formulations 3, 4 and 5 were as effective for preventing or alleviating cramps and preventing the reoccurrence of cramps. The results further demonstrate that a composition containing a combination of any of the active compounds (e.g., ATP, CoQ10, lipoic acid, and MCFAs), further combined with an analgesic, essential oil and/or penetration enhancer compound were effective; i.e., ATP supplementation and/or the generation of ATP where comparable to that of creatine alone for the regeneration of ATP.

Definitive formulations, compositions and componentry will be defined using biometric studies with different demographics, patients and muscle conditions as described above.

What is claimed:

1. A method of treating muscles, the method comprising:
applying a topical composition to an area of an individual's body; and
generating newly synthesized adenosine triphosphate (ATP) in a muscle of the individual to reduce or prevent muscle issues.

2. The method of claim 1 further comprising delivering ATP to the muscle via the topical composition.

3. The method of claim 1 further comprising boosting regeneration of ATP in the muscle from de novo synthesis of intracellular factors.

4. The method of claim 1 further comprising mitigating adverse effects in the muscle.

5. The method of claim 1 wherein the topical composition comprises:
a predetermined amount of middle (mid) chain fatty acid (MCFA); and
at least one compound selected from the group consisting of Acetyl Coenzyme A (Acetyl-CoA), adenosine triphosphate (ATP), amino acids (arginine, cysteine, glutamine, glycine, histidine and/or L-methionine or other amino acids), carnosine, citrate, Coenzyme Q10 (CoQ10), collagen, Flavin Adenine Dinucleotide (FADH₂), fumarate, glycogen, glucose, ketone bodies (acetoacetate and/or β-hydroxybutyrate), lactate/lactic acid, lipoic acid/liponate, malate, beta alanine, nicotinamide adenine dinucleotide (NADH), potassium (K), pantothenic acid (vitamin B5), pyruvate, and/or succinate.

6. The method of claim 1 wherein the muscle issues can be muscle cramps, muscle stiffness, muscle pain, or muscle spasms.

7. The method of claim 5 wherein the MCFA is selected from the group of a C6 saturated fatty acid, a C8 saturated fatty acid, a C10 saturated fatty acid and a C12 saturated fatty acid.

8. The method of claim 5 wherein the MCFA is present in the composition in an amount in a range of from about 0.01 percent to about 90 percent of the weight or volume of the composition.

9. The method of claim 5 wherein the MCFA is present in the composition in an amount in a range of from about 0.01 percent to about 40 percent of the weight or volume of the composition.

10. The method of claim 5 wherein the MCFA is present in the composition in an amount in a range of from about 0.01 percent to about 20 percent of the weight or volume of the composition.

11. The method of claim 5 wherein the MCFA is present in the composition in an amount in a range of from about 1 percent to about 10 percent of the weight or volume of the composition.

12. The method of claim 7 wherein the MCFA is a combination of the C8 saturated fatty acid and the C12 saturated fatty acid.

13. The method of claim 7 wherein the MCFA can be a salt of a C6 saturated fatty acid, a salt of a C8 saturated fatty acid, a salt of a C10 saturated fatty acid or a salt of a C12 saturated fatty acid.
